(19) United States
(12) Patent Application Publication
(10) Pub. No.: US 2013/0213415 A1
(43) Pub. Date: Aug. 22, 2013

(54) Para-meatal/stomal Adhesive Sealing Device

(71) Applicants: Dennis M. Kay, Tampa, FL (US); William Wilson, Plant City, FL (US)

(72) Inventors: Dennis M. Kay, Tampa, FL (US); William Wilson, Plant City, FL (US)

(21) Appl. No.: 13/402,494

(22) Filed: Feb. 22, 2012

(51) Int. Cl. A61F 5/48

(52) U.S. Cl. 128/885

(57) ABSTRACT

A para-meatal/stomal adhesive sealing device is formed of a layer of adhesive material, such as hydrocolloid, having a central body portion with an opening/aperture therein for alignment with a meatus or stoma and a plurality of leaves extending from the central body portion having parabolic curved sections at the adjoining portions thereof adjacent the central body portion.

### BACKGROUND OF THE INVENTION

1. Field of the Invention

The present invention relates to optimal para-meatal/stomal adhesive sealing devices to reduce leakage of bodily fluids and, more particularly, to an adhesive flat sheet having the capability to conform to convex surfaces.

2. Brief Discussion of the Related Art

Prior art adhesive devices for use with para-meatal/stomal skin surfaces to minimize leakage of bodily fluids and to secure catheters to the skin surfaces or in the body have, in many cases, still provided paths for leakage. As shown in U.S. Pat. No. 5,263,947 to Kay, such devices for use in collecting urine have utilized leaves or petals, as shown in FIGS. 1, 2 and 4 herein, where the leaves 3 have a vertex 15 at an acute angle between the leaves where each leaf joins a central body 6 having an opening 12 at a center point thereof for alignment with the meatus or stoma to which the device is applied. A catheter 8 typically communicates with the meatus via opening 12. As shown in FIG. 1, each leaf has a center line 14. Use of the devices shown in FIGS. 1 and 3 is shown in FIGS. 4 and 2, respectively, applied to a glans penis. Applicants have discovered that when the leaves are attached to the convex skin surface, a phenomenon, referred to as “tenting,” occurs wherein portions of the leaves of the device extending from the faceplate (formed by the central body and the leaves), adhere to adjacent portions of the faceplate rather than adhering to the tissue at the intended application site on the surface of the glans. The “tenting” is shown in FIG. 4 at 13. For the purposes of the present application, “tenting” is defined as portions of adjacent leaves that adhere to each other or overlap at the leaf vertices, rather than sealing to the surface of the glans penis. The “tenting” area is an accumulation of a raised ridge of material that fails to seal to the intended application surface. “Tenting” may occur at multiple vertices, decreases skin contact area and may propagate a leak path allowing migration of bodily fluids to leak between the leaves and/or the skin surface of the application site. Such leakage decreases the effective device wear time, it being noted that duration of leak-free wear time is a defining factor of a para-meatal/stomal adhesive sealing device's effectiveness.

### SUMMARY OF THE INVENTION

Applicants have, surprisingly, determined that leakage of bodily fluids can be limited by reducing the amount of adhesive material adjacent the leaf vertices. This is counterintuitive to the conventional technique in many fields of increasing the area of an adhesive layer to reduce leakage.

Accordingly, a primary aspect of the present invention is to achieve more effective sealing of a para-meatal/stomal adhesive device to an application site and to improve duration of use of the device.

In another aspect, the present invention eliminates the “tenting” phenomenon occurring in the prior art.

In a further aspect, a para-meatal/stomal adhesive sealing device according to the present invention is designed for adhesive skin attachment producing an adhesive seal optimized by contact of the adhesive seal with the para-meatal/stomal skin and wherein gaps or wrinkles in the adhesive seal that can decrease skin contact area are obviated such that no “leak path” is propagated that allows for the migration of bodily fluids between the adhesive layer and the skin surface which would cause a decrease in potential optimal wear time and adhesive holding capacity.

### BRIEF DESCRIPTION OF THE DRAWINGS

FIG. 1 is a plan view of a prior art para-meatal/stomal adhesive sealing device.

FIG. 2 is a perspective view of a prior art para-meatal/stomal adhesive sealing device fixed to a glans penis.

FIG. 3 is an isometric view of another prior art para-meatal/stomal adhesive sealing device.

FIG. 4 is an isometric view of a prior art para-meatal/stomal adhesive sealing device affixed to a glans penis and illustrating “tenting.”

FIG. 5 is a plan view of a faceplate of a para-meatal/stomal adhesive sealing device according to the present invention.

FIG. 6 is an isometric view of the para-meatal/stomal adhesive sealing device of FIG. 5 affixed to a glans penis.

FIG. 7 is an isometric view of the para-meatal/stomal adhesive sealing device according to the present invention in combination with seals, a catheter and a collection chamber.

### DESCRIPTION OF THE PRESENT INVENTION

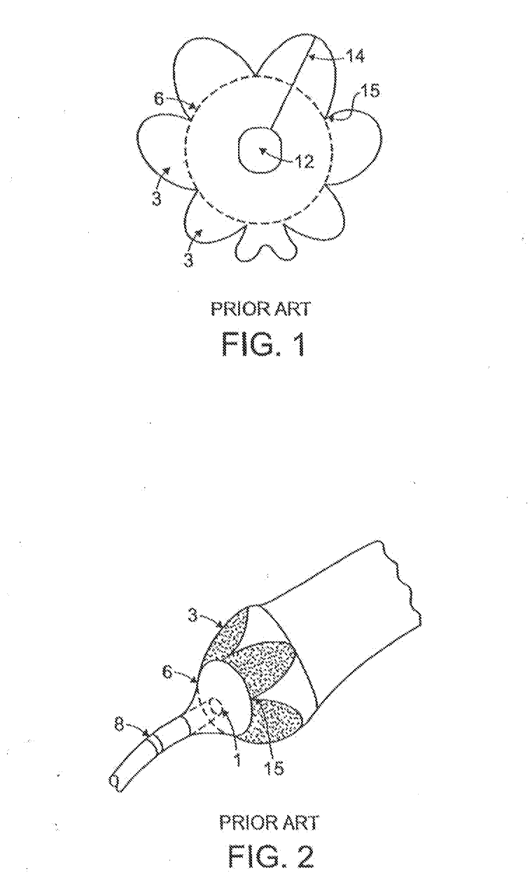

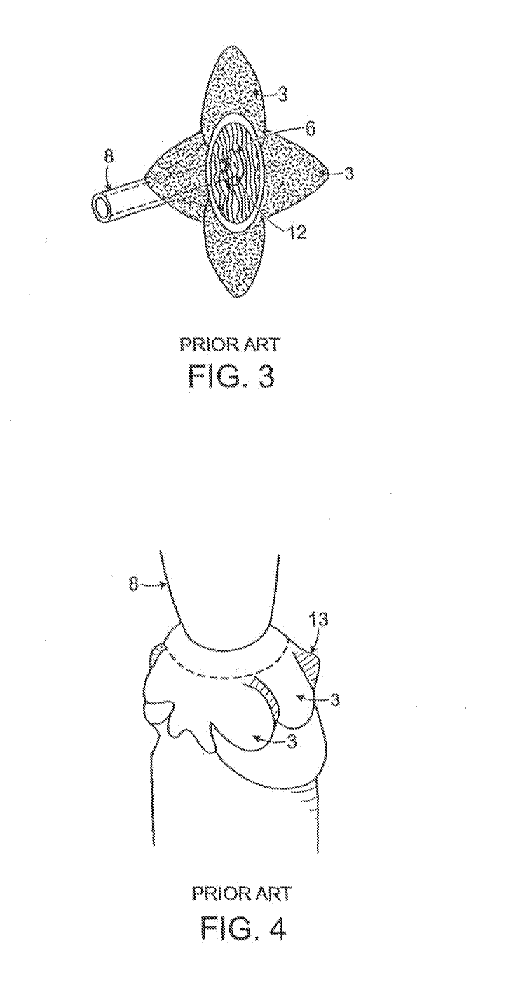

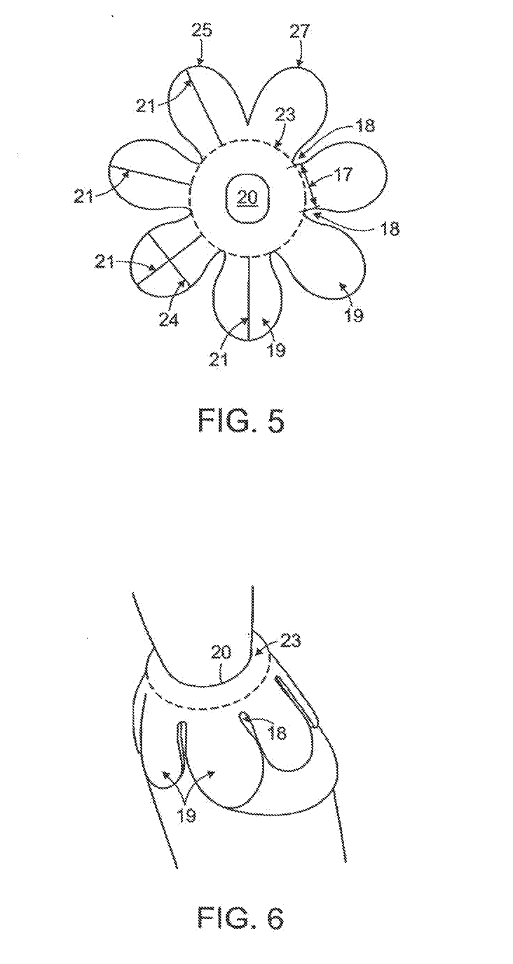

A para-meatal/stomal adhesive seal according to the present invention is shown in plan view in FIG. 5 in the form of a faceplate formed of a hydrocolloid adhesive layer including a plurality of leaves 19 extending from a circular central body portion 23. An aperture 20 is centrally located in the body portion for alignment with a meatus or stoma to allow passage of body fluids. The leaves 19 connect with the central body via a parabolic curved section 18 replacing the acute vertex 15 shown in the prior art device in FIG. 1. Lines 21 denote respective center lines for the leaves 19, and lines 17 denote a base leaf width with the line 17 perpendicular to the center lines 21 of the leaves at the base of the leaves where each leaf attaches to the central body 23. Lines 24, perpendicular to the center lines 21 at the widest parts of the leaves, define a maximum perpendicular width line 24. The surface area of each leaf proximal to the maximum perpendicular width line 24 is smaller than the surface area of each leaf distal to the maximum perpendicular width line 24. Accordingly, the sections 18 form gaps having curved edges adjacent the perimeter of the central body.

FIG. 6 is an isometric view of the adhesive sealing device of the present invention affixed or applied to a glans penis. It will be appreciated from FIG. 6 that, due to the configuration of the leaves and the parts of the leaves adjacent the central body 23, no “tenting” occurs.

In accordance with the present invention, “tenting” is eliminated by reducing the surface area of the proximal portion of the leaves 19 at the vertices. The acute angle vertices of the prior art are replaced with a parabolic arched portion defining the proximal margins of the leaves where the leaves become contiguous with the central body 23. The parabolic arcs define the proximal margins of the leaves such that, with respect to the central body, the minimum perpendicular leaf width 17 is proximal to the maximum perpendicular leaf width 24. In use, the side edges of adjacent leaves are not contiguous such that there is no overlap due to abutting leaves. Some of the leaves can have configurations to be diverging as shown at 25 and 27 with the side edges thereof meeting at an intersection spaced from the perimeter of the central portion.

The reduction in width of the proximal portion of the leaves 19 allows the leaves to conform more completely to the intended application site due to the decreased flexural modulus of the proximal portion of the structure of each leaf and the adhesive material as compared to the wider more distal portions of each leaf. Also due to the more complete conformation of each leaf to the intended application surface, “tenting” is avoided such that the adhesive sealing device of the present invention is less likely to be dislodged by penile retractions or the impact of adjacent tissue or objects against the sealed edges of the leaves 19.

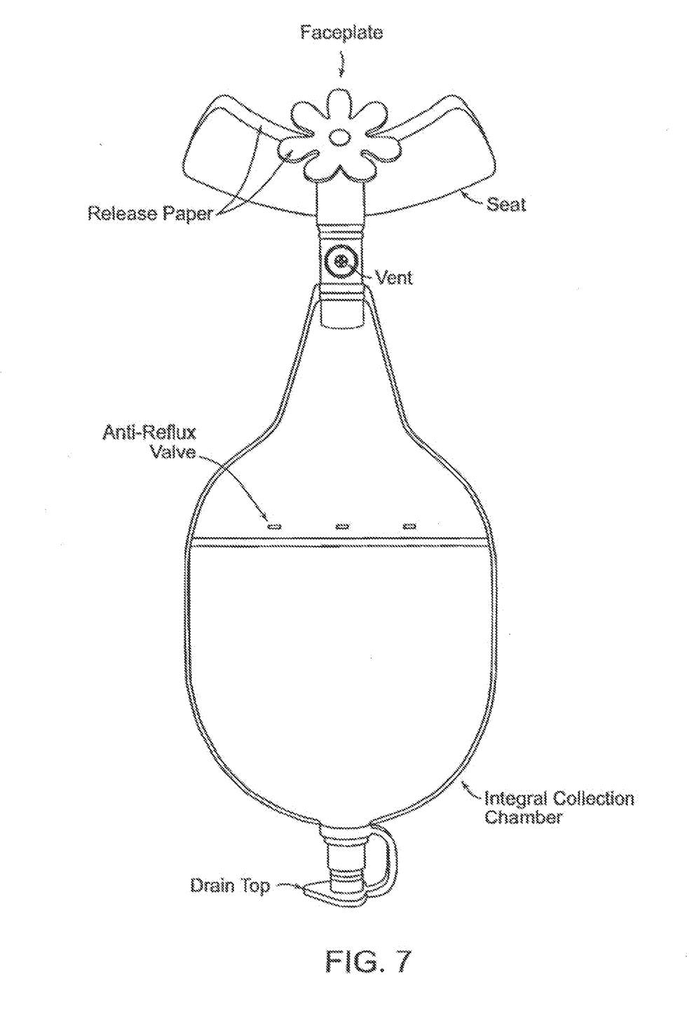

A system utilizing the faceplate is shown in FIG. 7 wherein it can be seen that the faceplate is provided adjacent seals and the catheter extending therethrough to receive urine from a urethral meatus communicates with an integral collection chamber.

While the present invention has been described and shown for use with a glans penis and the para-meatal surface thereof, the para-meatal/stomal adhesive sealing device of the present invention can be utilized for various stoma structures including, but not limited to, PEG tube ostomies, nephrostomies, and uretherostomies. To this end, it is noted that the para-meatal/stomal adhesive sealing device of the present invention can be used in areas represented as a truncated cone.

Inasmuch as the present invention is subject to many variations, modifications and changes in detail, it is intended that all subject matter discussed above or shown in the accompanying drawings be interpreted as illustrative only and not be taken in a limiting sense.

What is claimed is:

1. An adhesive sealing device for application to the human body at a para-meatal/stomal location comprising an adhesive layer defining a central portion with an aperture therein for alignment with the meatus/stoma to allow passage of body fluids therethrough, and a plurality of leaves extending substantially radially from said central portion, at least some of said leaves having arcuate vertices adjacent said central portion whereby the side edges of said leaves adjacent ones of said leaves are not contiguous.

2. The adhesive sealing device recited in claim 1 wherein the adhesive material is hydrocolloid.

3. The adhesive sealing device recited in claim 1 wherein the leaves have configurations such that portions of the leaves proximal to the central portion have a width less than the maximum width of the leaves.

4. The adhesive sealing device recited in claim 1 wherein said device further includes additional leaves having a configuration such that pairs of said additional leaves diverge from each other.

5. The adhesive sealing device recited in claim 1 wherein said arcuate vertices have a parabolic shape.

6. The adhesive sealing device recited in claim 1 wherein said arcuate vertices form gaps having curved edges adjacent the perimeter of said central portion.
